(19) United States
(12) Patent Application Publication
(10) Pub. No.: US 2015/0328162 A1
(43) Pub. Date: Nov. 19, 2015

(54) PHARMACEUTICAL PREPARATIONS

(71) Applicant: Mario Medri, Newark, NJ (US)

(72) Inventor: Mario Medri, Newark, NJ (US)

(21) Appl. No.: 14/278,772

(22) Filed: May 15, 2014

(51) Int. Cl. A61K 9/20; A61K 31/137; A61K 31/485; A61K 9/00; A61K 31/4402; A61K 31/225; A61K 31/4439; A61K 31/616; A61K 31/09

(52) U.S. Cl. CPC A61K 9/2013; A61K 31/616; A61K 31/137; A61K 31/485; A61K 31/09; A61K 31/4402; A61K 31/225; A61K 31/4439; A61K 9/0056

(57) ABSTRACT

The present invention relates to an improved pharmaceutical preparation comprising a dosage form of an active ingredient such as aspirin, acetaminophen, ibuprofen, naproxen sodium salt, or other NSAID, in the form of an easily chewable tablet, which when chewed is rapidly absorbed into the blood stream by the intimate presence of citric acid to yield desirably fast high blood levels.

### FIELD OF THE INVENTION

This invention relates to improved pharmaceutical preparations and more particularly to chewable pharmaceutical preparations that provide rapid absorption into the blood stream.

### BACKGROUND OF THE INVENTION

Various types of tablets and other delivery vehicles exist for oral administration of active ingredients and mediations. For example, aspirin is widely employed as an analgesic, antipyretic, and as an anti-coagulant for prevention of thrombosis, which may cause heart disease or strokes. Other Non-Steroidal Anti-Inflammatory Drugs (NSAIDs) such as ibuprofen, naproxen, and acetaminophen also are recommended or prescribed as a treatment for rheumatoid arthritis.

Applied pharmaceutical research has provided numerous dosage forms of pain relievers designed to fill particular needs. Ordinary tablets are suitable tor older children and adults capable of swallowing a pill with a mouthful of water. Sustained action tablets are also available which release an active ingredient gradually so that therapeutically satisfactory levels thereof are maintained in the blood stream over an extended period of time. For those who cannot swallow a tablet, a chewable form may be desirable. A chewable tablet which does not require water or other liquid for administration presents other challenges since the active ingredient may have an unpleasant and lingering taste. For example, as discussed in U.S. Pat. No. 3,567,819, the taste of aspirin in a chewable tablet can be masked by utilizing flavoring agents such as anise or licorice, or fruit flavors such as cherry or wild cherry. Making aspirin into a confectionary item creates other problems. Young children may mistake the aspirin tablets for candy causing them to consume sufficient tablets to cause an overdose or side effects. Accordingly, if an acceptable chewable tablet is to be formulated, the unpleasant taste of the active ingredient should be masked in a manner that will not invite children to consume the medicine as if it were candy. It is thereftre desirable to create a delivery system for delivering pain relief, anti-inflammatory, and antihistamine medication that has desirable taste characteristics, while providing rapid absorption in the blood stream.

### SUMMARY OF THE INVENTION

The foregoing disadvantages of prior drug delivery systems are overcome and the objects of the invention are accomplished by providing a chewable tablet having an active ingredient agglomerated with citric acid and then formulated into a chewable tablet as discussed herein.

The present invention also provides a method of manufacture of a chewable drug delivery tablet which comprises providing an active ingredient such as an anti-inflammatory, a NSAID, an anti-pyretic, an anti-coagulant, an antihistamine or other active ingredient typically administered orally; agglomerating particles of the active ingredient with citric acid to form agglomerated active ingredient crystals, and then formulating a chewable tablet containing the agglomerated citric acid and active ingredient crystals.

### BRIEF DESCRIPTION OF THE PREFERRED EMBODIMENTS

Further features and advantages of the invention will become apparent upon review of the following detailed description of preferred embodiments taken in conjunction with the following drawings, in which:

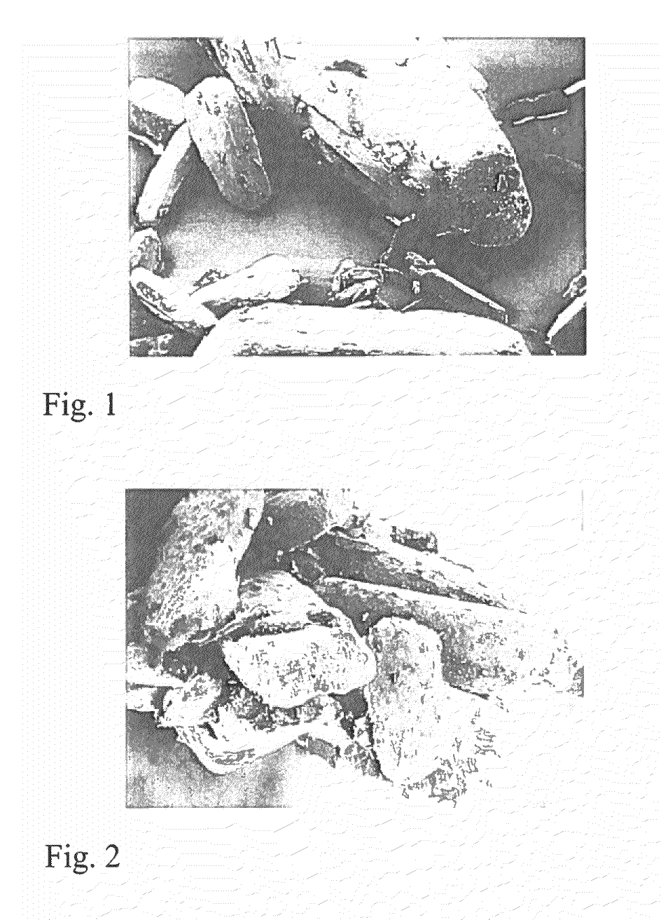

FIG. 1 shows uncoated aspirin particles at 240× magnification;

FIG. 2 is a 200× magnification of aspirin particles coated with 6% citric acid;

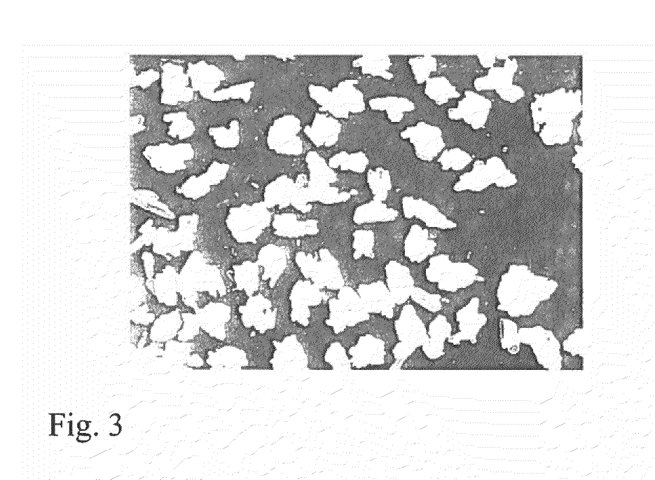

FIG. 3 is a 16× magnification of agglomerated aspirin particles;

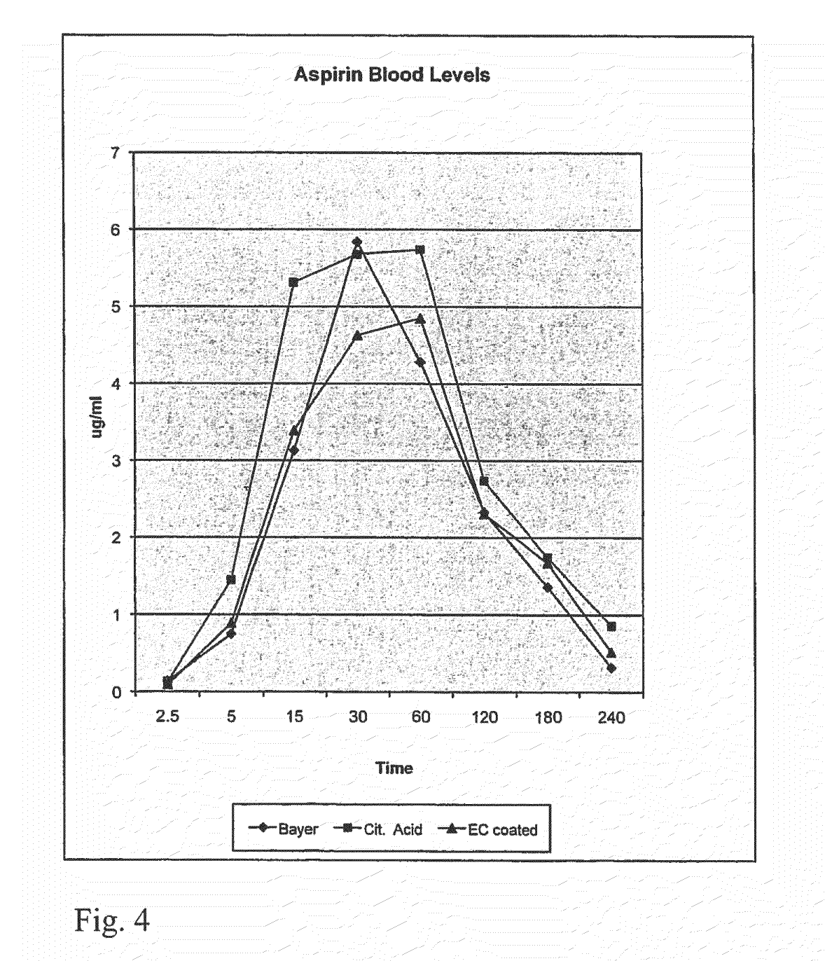

FIG. 4 is a graph of aspirin blood levels showing μg/ml vs. time after dosage of 5 grains of aspirin in accordance with the present invention, as compared to controls of an enteric coated aspirin tablet and a brand name aspirin tablet, the data of Table I;

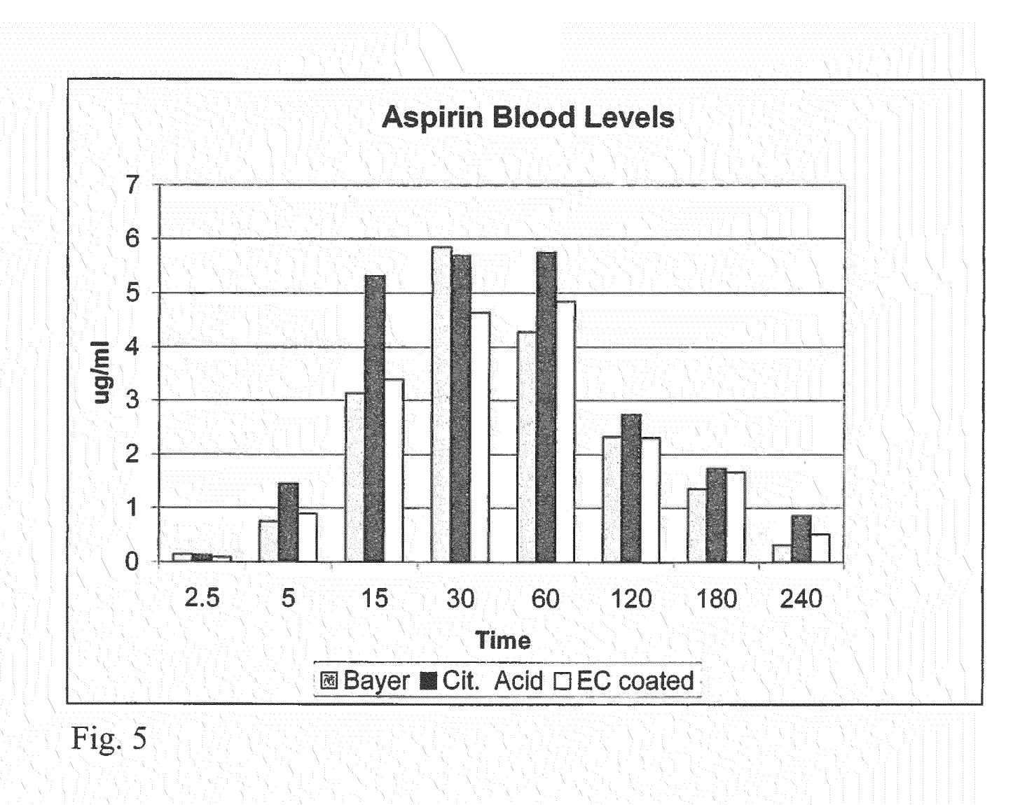

FIG. 5 is a bar graph of the same data from Table I comparing aspirin blood levels after dosages of a brand name aspirin tablet, a chewable tablet of the present invention, and an enteric coated aspirin tablet;

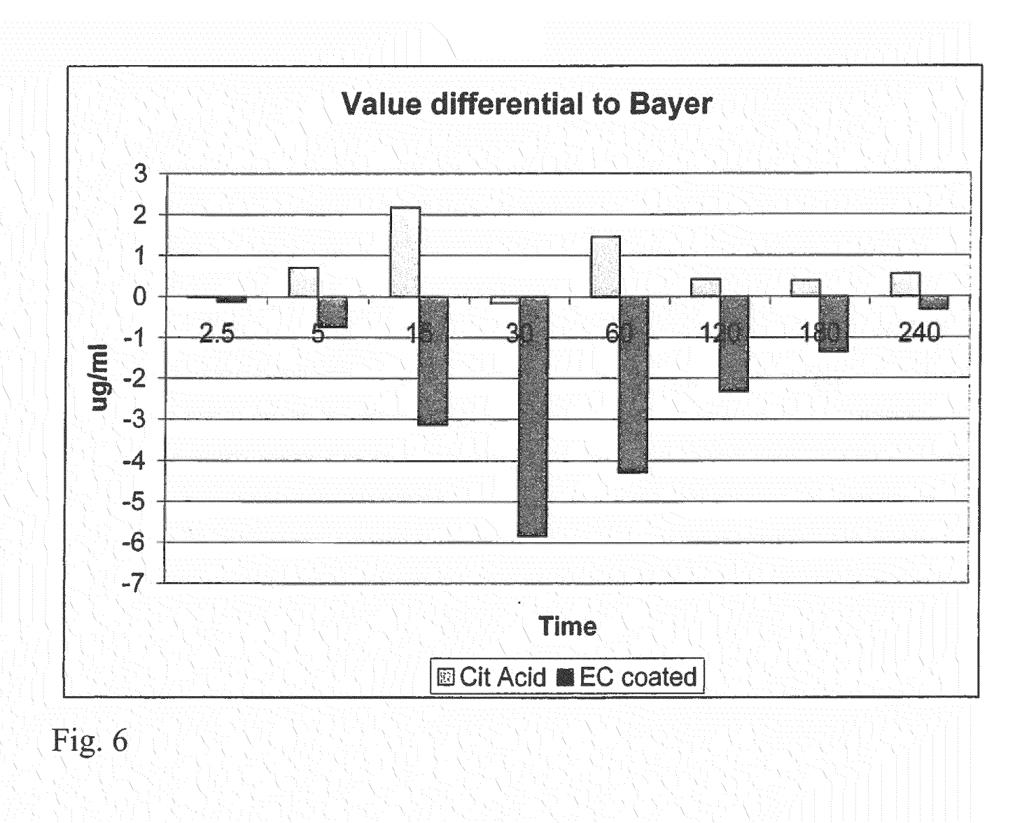

FIG. 6 is a graph showing differential value of citric acid coated aspirin versus enterically coated aspirin showing the data in Table I;

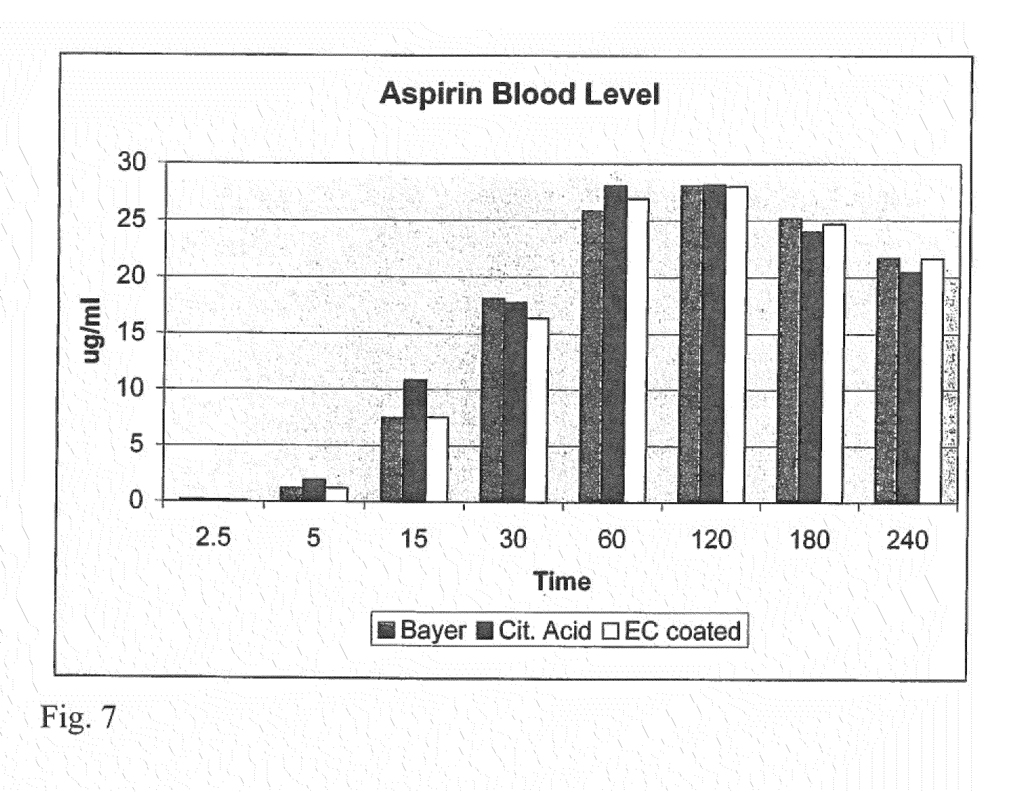

FIG. 7 is a bar graph comparing blood levels of aspirin formulated according to present invention to a commercially available aspirin tablet and an enteric coated aspirin tablet, using the data shown in Table II; and

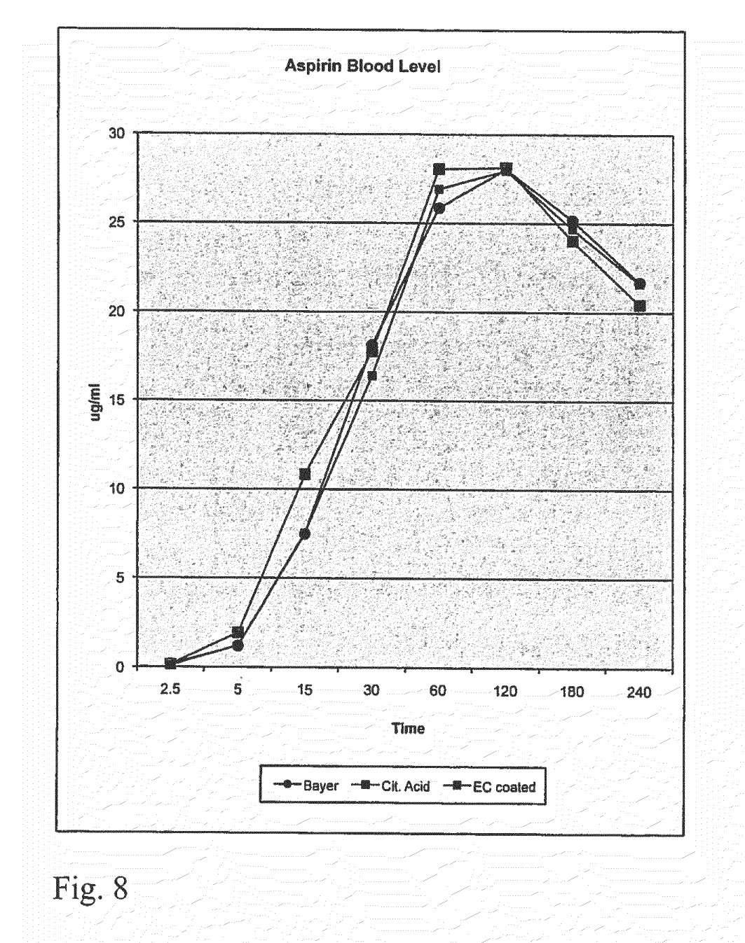

FIG. 8 is a line graph of the data shown in Table II and FIG. 10.

### DETAILED DESCRIPTION OF THE PREFERRED EMBODIMENTS

The invention provides a chewable pharmaceutical delivery tablet comprising an analgesic, anti-inflammatory, an antihistamine, a decongestant, a cough suppressant, expectant, a laxative, or an antacid or anti-pyretic pharmaceutically active ingredient agglomerated with citric acid wherein the citric acid is from about 5 to about 15% by weight on a dry basis of the pharmaceutically active ingredient. The agglomerated pharmaceutically active ingredient is in admixture with from about 80 to about 85% by weight of sorbitol, mannitol, isomalt, lactose, starch, stearic acid, magnesium stearate, talc, sucrose or a combination thereof as tableting excipients. The invention further provides a chewable tablet wherein the citric acid agglomerated pharmaceutical active ingredient comprises about 17.5% by weight of the tablet, and the tableting excipients compromise about 73.5% by weight mannitol, about 7% by weight starch and about 2% by weight stearic acid or magnesium stearate. In one embodiment, the pharmaceutically active ingredient is aspirin, acetaminophen, naproxen sodium salt, or ibuprofen. In another embodiment, the pharmaceutically active ingredient is aspirin with particle size ranging from about 60 to about 140 mesh, acetaminophen, or ibuprofen.

In a further embodiment, the pharmaceutically active ingredient is an antihistamine decongestant, expectorant, antacid, cough suppressant, or laxative. Where the chewable tablet is an antihistamine, the antihistamine is dichloropheniramine maleate, or it could be loratadine, cetirizine, fexofenadine, diphenhydramine or doxylamine. If the chewable tablet is a decongestant is pseudoephedrine. If the active ingredient is a cough suppressant, and the cough suppressant is dextromethorphan. If the active ingredient is an expectorant, the expectorant is guaifenesin, while if the active ingredient is a laxative, the laxative is bisacodyl or docusate. If the active ingredient is an antacid, the active ingredient is omeprazole.

The present invention is described herein with examples and formulations below relating to aspirin, it being understood that the invention can be practiced with numerous other prescription and non-prescription active ingredients, including without limitation acetaminophen, ibuprofen, naproxen sodium, anti-pyretics, anti-coagulants, antihistamines, including loratadine, cetirizine, fexofenadine, dichloropheniramine maleate and other NSAIDs, diphenhydramine, and doxylamine, among others; laxatives, including bisacodyl and ducosate; cough suppressant, including dextromethorphan; expectorants, including guaifenesin and antacids, including omeprazole, a proton pump inhibitor.

To agglomerate the aspirin crystals or particles of aspirin (ranging from about 60 to about 140 mesh) and in particular case of about 80 mesh (see FIGS. 1-3) with citric acid, the citric acid employed is first dissolved in water to form a citric acid saturated aqueous solution and thus obtained gradually added to the aspirin in suitable mixer. When it has all been added and then thoroughly mixed together the wet aspirin crystals are fast dried, preferably by means of a fluidized bed dryer. These agglomerated aspirin crystals differ both chemically and physically from a mere admixture of aspirin crystals and citric acid. Since the aspirin crystals have but a limited solubility in water they are substantially unaffected by the saturated aqueous citric acid solution with which they are in contact. Upon drying, the evaporation of the water yields agglomerates of coated crystals of aspirin with citric acid.

The amount of citric acid added to the aspirin to form the desired agglomeration of citric acid coated aspirin can vary. However, from about 5 to about 15% by weight, the preferably about 5 to about 10% by weight, yields very satisfactory results in masking the usually unpleasant aspirin taste. Thus, the desired amount of citric acid used in the coating/agglomeration of aspirin ranging in particle size from 60 to 140 mesh, may be dissolved in sufficient water at a suitable temperature to make a 50 to 65% by weight aqueous solution and this solution is the solution which is then added to the 80 mesh aspirin with mixing used in this particular case.

The following examples are meant to be an illustrative but not limiting disclosure of the practices of the invention using aspirin at 80 mesh particle size.

### Example 1

67.5 grams of anhydrous citric acid are added to 45 grams of distilled water at room temperature and the mixture stirred until solution is complete. This solution is then added to 1000 grams of 30 mesh crystalline aspirin (also applicable to aspirin with particle size up to 140 mesh) and the mixture stirred until it is substantially homogenous. The resulting mixture is then dried in a fluidized bed dryer.

### Example 2

A chewable aspirin tablet is prepared by combining the following ingredients:

| | Parts by Weight |
|---|---|
| Aspirin/citric acid (from Example 1) | 17.5 |
| Starch | 7 |
| Stearic acid | 2 |
| Mannitol (granular) | 73.5 |

The aspirin/citric acid is blended with one-half of the mannitol. The remaining mannitol, starch and stearic acid are then thoroughly blended in by mixing for 5 minutes. The mixture is then tableted into ¾ inch flat-faced bevel edged tablets weighing about 2 grams each to a hardness of 10 kg and having a thickness of about 0.235 inches. Each tablet contains 5 grains of aspirin.

The bioavailability of the aspirin in the chewable tablets of Example 2, as compared to the same amount of aspirin in Bayer aspirin tablets and in tablets formed of 2% ethyl cellulose encapsulated aspirin was determined in 12 adult subjects, two female and ten male, ranging in age from 21 to 49. After a routine physical examination all were found to be in good health with negative histories of gastro-intestinal, renal, cardiac and liver disease.

The above study was run in a double-blind, crossover design. Thirteen volunteers began the study, one dropped out before the completion of the study; his results are not included in the final analysis. The subjects were divided into three groups. In the above study the three groups received the test drugs at one week intervals according to the following design, the tablets being chewed and swallowed without any accompanying water (EC=ethyl cellulose).

| | Week 1 | Week 2 | Week 3 |
|---|---|---|---|
| Group | Bayer Aspirin | Tablets prepared | Tablets prepared with 2% |
| I | Tablets | with Citric Acid | EC Encapsulated Aspirin |
| | | Agglomerated | Powder |
| | | Aspirin Powder | |
| Group | Tablets prepared | Tablets prepared | Bayer Aspirin Tablets |
| II | with | with 2% EC | |
| | Citric Acid | Encapsulated Aspirin | |
| | Agglomerated | Powder | |
| | Aspirin | | |
| | Powder | | |
| Group | Tablets prepared | Bayer Aspirin | Tablets prepared with |
| III | with 2% EC | Tablets | Citric Acid |
| | Encapsulated | | Agglomerated Aspirin |
| | Aspirin Powder | | Powder |

The 2% ethyl cellulose encapsulated aspirin powder employed above is prepared in accordance with the procedures taught in the U.S. Pat. Nos. 3,155,590 and 3,341,416 in which an appropriate amount of ethyl cellulose is employed to yield the desired amount of ethyl cellulose in the encapsulated film.

On each experimental day the subjects had blood drawn from the antecubital vein in 10 cc. 3200 KA heparinized vacutainer tubes prior to and at 2.5, 5.0, 15, 30, 60, 180 and 240 minutes after receiving a 10 gr. dose of the medication. All of the blood samples drawn from the subject were processed immediately by pipetting 1.0 ml of whole blood into a test tube containing 8.0 ml of a 5% w/v HgCl₂ solution. The test tube was first placed on a Lab-Line Instrument Super-Mixer for immediate distribution of the whole blood throughout the HgCl₂ by the vortex during the pipetting of the whole blood. The solution was centrifuged in an International Clinical centrifuge Model CL at 2800 rpm for 5 minutes. A 3.0 ml aliquot of the supernatant liquid was treated with 2.0 ml 1 N NH₄OH in order to hydrolyze the conjugated salicylates. The ammoniacal sample was centrifuged at 2800 rpm for 5 minutes and then filtered through No. 3 Whatman filter paper into a G.S. test tube. The remaining unhydrolyzed HgCl₂ solution was also filtered through No. 3 Whatman filter paper into a G.S. test tube.

The fluorescence of each sample was measured on a Farrand Optical Company. Inc., Spectrofluorometer, Catalog No. 10424 3B. The unhydrolyzed samples were measured in chorographical order in a 1.0 cm. quartz cell at 410 mμ when activated at 310 mμ. A 150 watt, xenon arc lamp in a Rudolph Instruments lamp housing was the source of excitation. The fluorescence of the hydrolyzed samples was also measured in the chronological order which they had been drawn and at 310 mμ/410 mμ.

A standard curve was prepared from the U.S.P. reference salicylic acid in freshly drawn blood on each study day. Duplicate specimen samples were assayed to determine the reproducibility of the methods for salicylic blood levels during the entire study. All glassware was cleaned with 20% nitric acid solution, washed with distilled water and finally rinsed with triple distilled water. No commercial detergents that may cause background fluorescence were used to clean the glassware. All stopcocks were Teflon to avoid contamination from lubricants. Both total and free Salicylic Acid were measured. Acetylsalicylic Acid was calculated from the difference (Acetylsalicylic Acid=total Salicylic Acid−Free Salicylic Acid).

In order to reach a more meaningful result, the test run also included commercial “Bayer” aspirin tablets, as indicated above. The results of these tests were as follows:

TABLE 1
Mean Acetylsalicylic Acid Blood Levels
μg/ml

| | | Tablets Prepared With | |
|---|---|---|---|
| | | Citric Acid | Tablets Prepared With |
| Time | Bayer Aspirin | Agglomerated Aspirin | 2% EC Encapsulated |
| (Minutes) | Tablet | Powder | Aspirin Powder |
| 2.5 | 0.14 | 0.13 | 0.09 |
| 5.0 | 0.75 | 1.45 | 0.89 |
| 15 | 3.13 | 5.31 | 3.39 |
| 30 | 5.84 | 5.68 | 4.63 |
| 60 | 4.28 | 5.74 | 4.85 |
| 120 | 2.33 | 2.74 | 2.31 |
| 180 | 1.36 | 1.74 | 1.67 |
| 240 | 0.32 | 0.86 | 0.52 |

EC = ethyl cellulose

TABLE 2
Mean Acetylsalicylic Acid Blood Levels
μg/ml

| | | Tablets Prepared With | |
|---|---|---|---|
| | | Citric Acid | Tablets Prepared With |
| Time | Bayer Aspirin | Agglomerated Aspirin | 2% EC Encapsulated |
| (Minutes) | Tablet | Powder | Aspirin Powder |
| 2.5 | 0.17 | 0.15 | 0.11 |
| 5.0 | 1.20 | 1.94 | 1.22 |
| 15 | 7.47 | 10.84 | 7.52 |
| 30 | 18.14 | 17.77 | 16.40 |
| 60 | 25.86 | 28.05 | 26.91 |
| 120 | 28.03 | 28.13 | 27.96 |
| 180 | 25.18 | 24.02 | 24.69 |
| 240 | 21.69 | 20.45 | 21.62 |

EC = ethyl cellulose

The foregoing results indicate very clearly that chewable tablets formed of citric acid agglomerated aspirin give aspirin and total salicylate blood level, which are generally higher than the levels reached in the same time period with either Bayer aspirin tablets or with tablets formed of aspirin encapsulated with 2% ethyl cellulose. Similarly, these blood levels are maintained at these higher levels for significant period with these test subjects.

All patents and other publications cited herein are incorporated herein by reference.

1. A chewable pharmaceutical delivery tablet comprising an analgesic, anti-inflammatory, an antihistamine, a decongestant, a cough suppressant, expectant, a laxative, or an antacid or anti-pyretic pharmaceutically active ingredient agglomerated with citric acid wherein the citric acid is from about 5 to about 15% by weight on a dry basis of the pharmaceutically active ingredient, said agglomerated pharmaceutically active ingredient being in admixture with from about 80 to about 85% by weight of sorbitol, mannitol, isomalt, lactose, starch, stearic acid, magnesium stearate, talc, sucrose or a combination thereof as tableting excipients.

2. A chewable tablet in accordance with claim 1, wherein the citric acid agglomerated pharmaceutical active ingredient comprises about 17.5% by weight of the tablet, and the tableting excipients comprise about 73.5% by weight mannitol, about 7% by weight starch and about 2% by weight stearic acid or magnesium stearate.

3. A chewable tablet in accordance with claim 2, wherein the pharmaceutically active ingredient is aspirin, acetaminophen, naproxen sodium salt, or ibuprofen.

4. A chewable tablet in accordance with claim 2, wherein the pharmaceutically active ingredient is aspirin with particle size ranging from about 60 to about 140 mesh, acetaminophen, or ibuprofen.

5. A chewable tablet in accordance with claim 2, wherein the pharmaceutically active ingredient is an antihistamine decongestant, expectorant, antacid, cough suppressant, or laxative.

6. A chewable tablet in accordance with claim 5, wherein the antihistamine is dichloropheniramine maleate.

7. A chewable tablet in accordance with claim 5, wherein the decongestant is pseudoephedrine.

8. A chewable tablet in accordance with claim 5, wherein active ingredient is a cough suppressant, and the cough suppressant is dextromethorphan.

9. A chewable tablet in accordance with claim 5, wherein active ingredient is an expectant, and the expectorant is guaifenesin.

10. A chewable tablet in accordance with claim 5, wherein the active ingredient is a laxative and the laxative is bisacodyl or docusate.

11. A chewable tablet in accordance with claim 5, wherein the active ingredient is omeprazole.

12. A chewable tablet in accordance with claim 5, wherein the active ingredient is an antihistamine, and the antihistamine is loratadine, cetirizine, fexofenadine, diphenhydramine or doxylamine.
